(19) United States
(12) Patent Application Publication
(10) Pub. No.: US 2025/0095799 A1
(43) Pub. Date: Mar. 20, 2025

(54) TELEMEDICINE METHODS FOR MANAGEMENT OF A HEALTH CONDITION

(71) Applicant: inHealth Medical Services, Inc., Los Angeles, CA (US)

(72) Inventors: Michelle Alencar, Orange, CA (US); Aubrey Jenkins, Bothell, WA (US); Johnnie Jenkins, Kirkland, WA (US)

(21) Appl. No.: 18/747,322

(22) Filed: Jun. 18, 2024

Related U.S. Application Data
(63) Continuation of application No. 17/545,948, filed on Dec. 8, 2021, now abandoned.
(60) Provisional application No. 63/122,761, filed on Dec. 8, 2020.

(51) Int. Cl. G16H 10/20; G16H 20/00; G16H 40/67; G16H 80/00

(52) U.S. Cl. CPC G16H 10/20; G16H 20/00; G16H 40/67; G16H 80/00

(57) ABSTRACT

A method for managing a health condition in a subject in need thereof by reinforced lifestyle adjustment based on one or more underlying motivations is described. The method includes obtaining a self-assessment for health-related categories. The method includes creation of a health vision plan for improving the health of the subject with respect to the health-related categories. The method includes identifying at least one action step for the subject to take in furtherance of achieving a short or long term goal. The method further includes re-evaluating the at least one action step using second information obtained from a meeting with the subject. The method includes providing messages that include content configured to encourage the subject, remind the subject of action steps, or educate the subject on the selected subset of health-related categories.

### PRIORITY

This application is a continuation of U.S. patent application Ser. No. 17/545,948, filed Dec. 8, 2021, which claims the benefit of U.S. Provisional Patent Application No. 63/122,761, filed Dec. 8, 2020, titled “TELEMEDICINE METHODS FOR MANAGEMENT OF A HEALTH CONDITION,” which is hereby incorporated by reference in its entirety.

### TECHNICAL FIELD

The present disclosure relates generally to a method for managing a health condition in a subject in need thereof by reinforced lifestyle adjustment based on one or more underlying motivations.

### BACKGROUND

There is an increased demand for effective healthcare solutions accessible remotely, such as telemedicine. Telemedicine refers to various activities to deliver care at a distance. With an increase in the amount of people who have or have access to smart phones (i.e., In 2017, an estimated 58% of adults owned a smart phone), healthcare is beginning to make a shift to online platforms, in which people are able to access healthcare without visits to a healthcare provider. Telemedicine technology has been shown to provide healthcare to locations that experience a lack in specialized services. See, e.g., Dorrian et al., Journal of Telemedicine and Telecare, “Head and Neck Cancer Assessment by Flexible Endoscopy and Telemedicine,” (2009); Givens & Elangovan, American Journal of Audiology, “Internet Application to Tele-Audiology,” (2003). Additionally, telehealth technologies have been shown to improve patient outcomes while lowering cost. See, e.g., Young & Ireson, Pediatrics, “Effectiveness of School-Based Telehealth Care in urban and Rural Elementary Schools,” (2003); Smith et al., Journal of Telemedicine and Telecare, “The Point-of-Referral Barrier,” (2001).

Recently, due to the novel coronavirus disease-19 (COVID-19) pandemic, telehealth has been rapidly adopted and implemented in order to deliver care at a distance. Activities such as remote patient monitoring, messaging, telephone visits, and video visits have moved entirely to distance care. Wosik, J., et al., JAMIA, “Telehealth Transformation: COVID-19 and the Rise of Virtual Care,” (2020). The move from in-person visits, to online appointments allows equal access to all patients regardless of location.

However, it has been found that switching from in-person care to telemedicine disincentivizes patients to be engaged. The switch to telemedicine makes it easier to cancel appointments remotely (e.g., “text cx2 to cancel”) and erases accountability between the health professional and the patient. For example, a recent study reported that while 21-88% of users were at least minimally engaged in a program after completing an initial assessment, only 0.5-29% were engaged at a last assessment or continued to be engaged after six weeks or more of a program. Fleming et. al., J. Med. Internet. Res., “Beyond the Trial: Systematic Review of Real-World Uptake and Engagement With Digital Self-Help Interventions for Depression, Low Mood, or Anxiety,” Vol 20, 6 e199 (2018). For example, an application for managing a post-traumatic stress disorder resulted in only 19.4% engagement after six months. Id. Often, this low engagement is in large due to the lack of the patient's ability to self-motivate.

For example, obesity is a major problem in the U.S., as an estimated 69% of adults have a body mass index (BMI) of greater than 30 kg/m². High fat mass in the body increases the risk of diabetes, hypertension, stroke, and cancer. The method of treating obesity has moved almost entirely to telemedicine applications.

Current telehealth platforms have the ability to count calories, and/or track physical exercise (e.g., step count). Additionally, current solutions may provide a recommended caloric intake and step count based on patient factors (e.g., age, weight, height, weight goal, etc.). For example, a patient that wants to lose 20 lbs. may have a recommended program to stay under 1700 calories daily and walk 10,000 steps a day.

However, there are several problems with this automated approach to treating obesity. Often, in order to see long-term results, a behavior modification is needed. Current platforms fail to incentivize a behavioral change. This is in large part due to a patient's lack of motivation to continue a program. Traditional telehealth treatment for obesity consists of an automated physical fitness and diet plan for the patient to complete. In order for the patient to successfully complete the program, the patient must have an understanding and intrinsic motivation to manage their own health and wellness, which is rare amongst patients who require a lifestyle change.

Currently, there is a need in the market for a solution to solve the lack of patient engagement in telehealth applications. To date, the current available technologies fail to individualize treatment for each subject, instead relying on automated health plans for the patient to follow without significant outside help or resources. Therefore, there is a need in the market for a telehealth care method and model that addresses the issue of patient engagement.

The information disclosed in this Background section is only for enhancement of understanding of the general background of the invention and should not be taken as an acknowledgment or any form of suggestion that this information forms the prior art already known to a person skilled in the art.

### SUMMARY

Given the above background, there is a need in the art for improved methods and systems for increasing engagement in telehealth applications. In particular, there is a need to incentivize patients to change their health condition and empower the patients to complete health programs. Advantageously, the present disclosure solves this and other needs in the art by providing an improved method that encourages patient engagement by allowing the subject to discern a health area that the subject would like to focus on. For example, a health professional consults with a patient about their health condition and the patient decides a health-related category that the patient would like to work on.

For example, in one aspect, the present disclosure provides a method for managing a health condition in a subject (e.g., patient) in need thereof by reinforced lifestyle adjustment based on one more underlying motivations.

The method includes obtaining, from the subject at a first time point, a first scored self-assessment for a plurality of health-related categories across a plurality of metrics, the first self-assessment including, for each respective health-related category in the plurality of health-related categories a first measure of the subject's assessment of their own health relative to the respective health-related category, a first measure of the subject's satisfaction with their own health relative to the respective health-related category, and a first measure of the subject's desire to improve their own health relative to the respective health-related category.

The method further includes selecting a first subset of health-related categories from the plurality of health-related categories based on the first scored self-assessment.

The method further includes facilitating subject creation of a health vision plan for improving the health of the subject with respect to the selected subset of health-related categories using first information arising from one or more meetings with the subject. The first information arising from one or more meetings with the subject comprises a description of the subject's desired health outcome elicited from the subject and one or more values, one or more personal experiences, one or more behaviors, or one or more motivations of the subject elicited from discussion with the subject.

The method then includes establishing a short term goal for improving the health of the subject, relative to the selected subset of health-related categories, over a first epoch, establishing a long term goal for improving the health of the subject, relative to the selected subset of health-related categories, over a second epoch, wherein the second epoch subsumes the first epoch and is at least twice as long as the first epoch. The short term goal and the long term goal create a path between the subject's current health and the subject's desired health with respect to the selected subset of health-related categories.

The method includes identifying one or more strategies the subject believes will be effective to achieve the short or long term goals and identifying, based on at least the one or more identified strategies, at least one action step for the subject to take in furtherance of achieving the short or long term goal.

Additionally, the method includes re-evaluating the at least one action step using second information obtained from a primary follow-up meeting with the subject, wherein the follow-up meeting occurs after one or more initial meetings and during the first and second epochs, and wherein the second information comprises a description of the subject's perception of their execution of the at least one action step elicited from the subject, a determination of whether the subject wishes to modify the long term goal, the short term goal, or the at least one action step, wherein when it is determined that the subject wishes to modify the long term goal, the short term goal, or the at least one action step, the long term goal, the short term goal, or the at least one action step is reestablished in modified form.

The method then includes re-evaluating the at least one action step during each of a series of secondary follow-up meetings with the subject, wherein the series of secondary follow-up meetings occur after the primary follow-up meeting and during the second epoch; and providing to the subject, outside of a meeting with the subject, one or more messages that include content configured to encourage the subject to achieve the long term goal or the short term goal, remind the subject of one or more of the at least one action step, or educate the subject on the selected subset of health-related categories.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### BRIEF DESCRIPTION OF THE DRAWINGS

The following figures are included to illustrate certain aspects of the embodiments, and should not be viewed as exclusive embodiments. The subject matter disclosed is capable of considerable modifications, alterations, combinations, and equivalents in form and function, as will occur to those skilled in the art and having the benefit of this disclosure.

FIG. 1 is a schematic chart illustrating the method for managing a health condition in a subject in need thereof by reinforced lifestyle adjustment based on one or more underlying motivations, in accordance with some embodiments.

FIG. 2 illustrates an example of a graphical user interface on the device of a user, in accordance with some embodiments.

FIG. 3 illustrates a progress tracking feature on a graphical user interface, in accordance with some embodiments.

FIG. 4 illustrates a messaging feature on a graphical user interface, in accordance with some embodiments.

FIG. 5 illustrates a profile feature on a graphical user interface, in accordance with some embodiments.

FIG. 6 illustrates a nutrition tracking feature on a graphical user interface, in accordance with some embodiments.

FIG. 7 illustrates a fitness tracking feature on a graphical user interface, in accordance with some embodiments.

FIG. 8 illustrates a water intake feature on a graphical user interface, in accordance with some embodiments.

FIG. 9 illustrates a supplement tracking feature on a graphical user interface, in accordance with some embodiments.

FIG. 10 illustrates a navigation feature on a graphical user interface, in accordance with some embodiments.

### DETAILED DESCRIPTION

The detailed description set forth below describes various configurations of the subject technology and is not intended to represent the only configurations in which the subject technology may be practiced. The detailed description includes specific details for the purpose of providing a thorough understanding of the subject technology. Accordingly, dimensions may be provided in regard to certain aspects as non-limiting examples. However, it will be apparent to those skilled in the art that the subject technology may be practiced without these specific details. In some instances, well-known structures and components are shown in block diagram form in order to avoid obscuring the concepts of the subject technology.

It is to be understood that the present disclosure includes examples of the subject technology and does not limit the scope of the appended claims. Various aspects of the subject technology will now be disclosed according to particular but non-limiting examples. Various embodiments described in the present disclosure may be carried out in different ways and variations, and in accordance with a desired application or implementation.

The present disclosure provides a method for managing a patient's health condition by reinforcement of lifestyle adjustments. The method involves identifying one or more underlying motivations that would drive the patient to make lifestyle adjustments that would support managing and/or improving the patient's health condition. The one or more underlying motivations are defined based on the patient's self-evaluation and with support of a health coach. The identified motivations are used as a basis for generating a health vision plan for the patient. The health vision plan includes defining short term and long term goals action steps that promote the lifestyle adjustments in accordance with the identified motivations. The provided method is believed to fill the existing void in the industry by providing an effective telemedicine approach for managing a health condition. More specifically, the present disclosure relates to coaching (e.g., promoting and supporting development) a subject to identify a category of health that they would like to improve, and provides them with the tools and support to ensure that they accomplish their goals.

Table 1 illustrates an exemplary step by step timeline for a six month coaching program for supporting a patient's lifestyle changes. In some embodiments, the program is a telemedicine program in that the coach and the patient use electronic forms of communications. For example, the coach and the patient meet via video conferences or teleconferences. As another example, the coach and the patient communicate via electronic service platforms (e.g., by e-mailing, text messaging, or via a mobile device application). In some embodiments, the coaching program is initiated during a “First visit” meeting (e.g., a videoconference meeting) during which the coach requests the patient to complete an initial wellness assessment, as illustrated in Table 1. In some embodiments, the initial wellness assessment is a self-assessment related to the patient's happiness and/or satisfaction level in regard to different focus health-related categories (e.g., physical health, mental health, nutritional health, etc.). For example, the patient is requested to assess (e.g., provide a quantitative score) her level of satisfaction with respect to the different health-related categories as well as importance of the health-related categories. For example, the patient provides a score between 0 to 10, where 0 corresponds to a low satisfaction and 10 corresponds to a high satisfaction toward the respective health-related category. In some embodiments, the coaching program is then continued, during the first month of the program, with an “Initial Wellness Assessment” meeting, as illustrated in Table 1. During the “Initial Wellness Assessment” meeting, the coach and the patient discuss the wellness assessment completed by the patient. In some embodiments, the coach guides the patient to identify, based on the initial wellness assessment, underlying motivations that could drive the patient to do make lifestyle changes for managing his or her health condition. As a result of the “Initial Wellness Assessment” meeting (e.g., during the first month of the program), the guide develops a “Health Vision Plan.” The “Health Vision Plan” includes a vision of the lifestyle changes that the patient is seeking to achieve to manage her health condition. In some embodiments, in accordance with the “Health Vision Plan,” the patient defines, guided by the coach, a set of goals and actions steps (e.g., SMART goals illustrated in Table 1) that enable and support the lifestyle changes identified in the “Health Vision Plan.” In particular, the set of goals and action steps include one or more short term goals (e.g., one or more 30 day goals) and one or more long term goals (e.g., one or more 6 month goals). The “Initial Wellness Meeting” is followed up by a plurality of follow up meetings during months 1-6 of the coaching program. In some embodiments, a follow up meeting is held once a week or once in every two weeks. In some embodiments, every 30 days during the coaching program, a follow up (“Milestone”) meeting includes assessing whether the one or more 30-day goals are met and assessing a status of the one or more 6-month goals. The assessing includes, e.g., assessing a need for revising or changing the goals and setting one or more new 30-day goals. In some embodiments, the follow up meeting also includes assessing a need for revising or changing the health-related categories identified in the “Health Vision Plan” that are most important to the patient. In alternate follow up meetings during the months 2 through 6, the patient is also requested to complete a program satisfaction survey or a wellness assessment survey. A program satisfaction survey provides an indication on how satisfied the patient is with the progress of the program (e.g., how likely the patient will engage with the program in the future). The repeated wellness assessment survey provides an indication on the progress the patient makes in regard to management of his or her health condition. Based on the program satisfaction surveys and/or the wellness assessment surveys, the goals and/or the “Health Vision Plan” may be changed or revised.

TABLE 1
Timeline for an example wellness management coaching program

| Visit | Coach Action Items | Materials for Coach | Materials for Patients |
|---|---|---|---|
| First visit | Before Visit: Review any clinical | Intro to coaching | Program Manual |
| | documents/referral from a physician. | script | Coaching Agreement |
| | During Visit: Request patient to complete | | Wellness Assessment |
| | a coaching agreement and an initial | | based on health-related |
| | wellness assessment. | | categories (e.g., 7 pillars) |
| | After Visit: Enroll the patient | | Introductory materials for |
| | to the program. | | health-related categories |
| MONTH 1 | | | |
| Initial | Before Visit: Review the patient’s | Initial Intake | SMART goals |
| Wellness | medical records and referral and score | Assessment script | Ideal Future Self |
| Assessment | wellness assessment. | Initial Intake | Relationship with Self |
| | Discuss during the visit: | Assessment script | Goal setting from the |
| | satisfaction scores | Intake scoring | inside out |
| | priority scale | sheet | Your WHY |
| | area of focus | Design a Vision | |
| | health vision | Health coach plan | |
| | health coaching plan | worksheet | |
| | After Visit: | | |
| | Provide the Health Vision Plan | | |
| | to the patient. | | |
| Follow Up | Before Visit: Review last visit’s notes and | Health coaching | Provider/reference |
| Visit | score wellness. | plan worksheet | education handouts as |
| | During Visit: Complete health coaching | Focus area | appropriate/requested |
| | plan including a 6-month goal, 30-day goal, | blueprints | |
| | and action steps. | | |
| | After Visit: Provide patient education to | | |
| | support patient’s goals, as | | |
| | needed/requested. | | |
| Follow Up | During Visit: Review action steps for 30-day | | |
| Visit | goal and area of focus. | | |
| MONTH 2 | | | |
| Follow Up | During Visit: Request the patient to | | Wellness satisfaction |
| Visit | complete a wellness satisfaction | | survey |
| | assessment. | | |
| | After Visit: Enroll patient in a wellness | | |
| | satisfaction survey. | | |
| Follow Up | Before Visit: Review the wellness | | |
| (Milestone) | satisfaction scores. | | |
| Visit | During Visit: Review the wellness scores | | |
| | with patient. | | |
| | Every 30-days: Check whether the 30-day | | |
| | goals are met, assess the need for revising | | |
| | the goals and/or area of focus. Review a | | |
| | status of the 6-month goal. Set new action | | |
| | steps. | | |
| MONTH 3 | | | |
| Follow up | During Visit: Request the patient to | | Program satisfaction |
| Visit | complete a program satisfaction survey. | | survey |
| | After Visit: Enroll the patient in a program | | |
| | satisfaction survey | | |
| Follow Up | Every 30-days: Check whether the 30-day | | |
| (Milestone) | goals are met, assess the need for revising | | |
| Visit | the goals and/or area of focus. Review a | | |
| | status of the 6-month goal. Set new action | | |
| | steps. | | |
| MONTH 4 | | | |
| Follow Up | During Visit: Request the patient to | | Wellness satisfaction |
| Visit | complete a program satisfaction survey. | | survey |
| | After Visit: Enroll patient in a program | | |
| | satisfaction survey | | |
| Follow Up | Every 30-days: Check whether the 30-day | | |
| (Milestone) | goals are met, assess the need for revising | | |
| Visit | the goals and/or area of focus. Review | | |
| | status of the 6-month goal. Set new action | | |
| | steps. | | |
| MONTH 5 | | | |
| Follow up | During Visit: Request the patient to | | Program satisfaction |
| Visit | complete a program satisfaction survey. | | survey |
| | After Visit: Enroll the patient in a program | | |
| | satisfaction survey | | |
| Follow Up | Every 30-days: Check whether the 30-day | | |
| (Milestone) | goals are met, assess the need for revising | | |
| Visit | the goals and/or area of focus. Review | | |
| | status of the 6-month goal. Set new action | | |
| | steps. | | |
| MONTH 6 | | | |
| Follow Up | During Visit: Request patient to complete a | | Program satisfaction |
| Visit | program satisfaction survey and a wellness | | survey |
| | assessment (re-assessment). | | Wellness assessment |
| | After Visit: Enroll patient in a program | | |
| | satisfaction survey and wellness | | |
| | assessment. | | |
| 6-Month | Before Visit: Review the wellness | | |
| Follow up | assessment scores. | | |
| (Milestone) | Every 30-days: Check whether the 30-day | | |
| Visit | goals are met, assess the need for revising | | |
| | the goals and/or area of focus. Check | | |
| | whether the 6-month goals are met. | | |

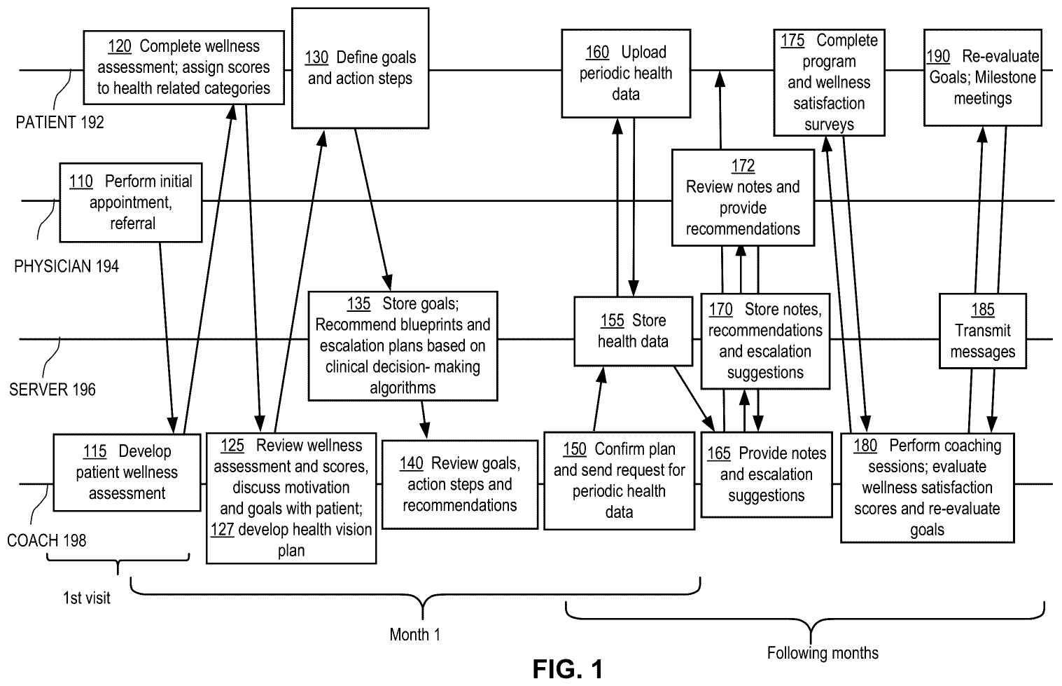

FIG. 1 is a chart illustrating the method for managing a health condition in a subject in need thereof by reinforced lifestyle adjustment based on one or more underlying motivations according to some embodiments. In some embodiments, method 100 comprises various steps for the patient 192, physician 194, server 196, and coach 198 to perform. In some embodiments, method 100 includes the coaching program described in Table 1 above.

In some embodiments, the method includes three time points (e.g., a first visit, a first month, following months). For example, as illustrated in Table 1 above, the program is carried out during a 6 month time period and includes the “First Visit,” three visits during the first month of the program including the “Initial Wellness Assessment” meeting, and a plurality of follow up meetings during months 2-6. In some embodiments, the time points are epochs. For example, a long term epoch corresponds to a 6 month time period and the 6 month time period includes several short term epochs (e.g., month 1, month 2, and month 3 in Table 1). In some embodiments, there are more than three time points. In some embodiments, the program is carried through a 3 month time period, a 6 month time period, a 9 month time period, a 12 month time period, an 18 month time period, or a longer time period.

In some embodiments, the physician 194 performs 110 an initial appointment with the patient 192. During the initial appoint, the physician 194 may diagnose the patient 192 with a health condition. In some embodiments, managing and/or treating the health condition would benefit from lifestyle changes. For example, in some embodiments, the health condition is obesity, diabetes, cardiovascular disease, or hypertension. In such embodiments, managing and/or treating the health condition would benefit from lifestyle changes that the patient could make. Such lifestyle changes may include, for example, losing weight, eating healthier, increasing amount or type of exercise, quitting smoking, reducing or eliminating consumption of alcohol or narcotics, reducing stress, etc. In some embodiments, the initial appointment with the physician 194 is an in-person meeting, or a virtual meeting (e.g., over the phone, or via a videoconference).

In some embodiments, physician 194 prescribes a medical treatment for managing and/or treating the health condition. In some embodiments, the treatment is a referral to participate in a health management program (e.g., a telemedicine based coaching program illustrated in Table 1) assisted by a health coach 198. The health coach may be a personal trainer, a therapist (physical, emotional, occupational, etc.), doctor, or other health professional. The prescription of a medical treatment may include information relating to the health of a patient 192. In some embodiments, the health information includes the patient's 192 medical record. In some embodiments, the health information is a current medical diagnosis. In some embodiments, the diagnostic information is sent through a server 196 to the health coach 198 (e.g., coach 198). In some embodiments, the prescribed medical treatment for managing and/or treating the health condition further includes medical treatment (e.g., drug based treatment).

As shown, the coach 198 receives the referral for assisting the patient 192 to manage and/or treat the health condition from the physician 194. In some embodiments, the coach 198 develops 115 an initial patient wellness assessment for the patient 192. In some embodiments, the initial wellness assessment is developed based on information relating to the patient's health condition. In some embodiments, the initial wellness assessment is based on information provided by the physician 194 in the referral (e.g., including the patient's medical diagnosis).

In some embodiments, the initial wellness assessment includes a plurality of questions configured for helping the patient 192 to self-assess her satisfaction toward a plurality of health-related categories. In some embodiments, the one or more health-related categories might be selected from a list comprising: physical health, nutritional health, emotional health, social health, environmental health, sense of purpose, and spiritual health.

In some embodiments, the health-related categories are referred to as pillars. For example, in Table 1 the patient receives materials for an “Initial Wellness Assessment” based on seven pillars during the “First Visit.” In some embodiments, seven pillars are: 1) physical health (e.g., how the patients fitness and movement affects her health), 2) nutritional health (e.g.,, how the patients nutrition affects her health), 3) emotional health (e.g., mental and emotional well-being), 4) environmental health (e.g., how the patient's immediate or greater environment supports her health), 5) social health (e.g., social connections including friendships, family, interactions with others), 6) sense of purpose (e.g., what are the purpose and passion of the patient), 7) spiritual health (e.g., the patient's connection in the world or a higher power). In some embodiments, the initial wellness assessment includes information relating to all of the seven pillars. In some embodiments, the initial wellness assessment relates to less than all seven pillars (e.g., from 4 pillars, from 3 pillars, from 2 pillars, or from 1 pillar).

In some embodiments, the initial patient wellness assessment is conducted by the patient 192. In some embodiments, the patient 192 chooses which of the seven pillars (all or less) they would like to complete the assessment for. In some embodiments, the physician 194 assigns the initial patient wellness assessment and discerns which of the seven pillars (all or less) to require the patient 192 to respond to. The initial patient wellness assessment may comprise a plurality of measures by the patient. For example, the patient 192 assigns a first measure of their own health relative to the respective health-related category. In some embodiments, the first measure is a quantitative measure. In some embodiments, the first measure is a qualitative measure. For example, the patient 192 assigns numerical values to one or more health categories based on how satisfied they are with the respective health category (e.g., a 1 is poor health, a 10 is excellent health).

In some embodiments, the patient wellness assessment includes a first measure of the patient's 192 satisfaction with their own health relative to the respective health-related category. In some embodiments, the first measure is a quantitative measure. In some embodiments, the first measure is a qualitative measure. In this example, the patient 192 may take a satisfaction assessment in which the patient 192 ranks their satisfaction of their health with one or more health categories (e.g., a 1 is not satisfied, and a 10 is satisfied).

In some embodiments, the patient wellness assessment includes a first measure of the patient's 192 desire to improve their own health relative to the respective health-related category. In some embodiments, the first measure is a quantitative measure. In some embodiments, the first measure is a qualitative measure. In this example, the patient 192 may take a satisfaction assessment in which the patient 192 ranks their desire to improve their own health with one or more health-related categories (e.g., a 1 is no desire, and a 10 is a great desire).

In some embodiments, the patient wellness assessment includes a first measure of importance of the respective health-related category. In some embodiments, the first measure of the importance is a quantitative measure. In some embodiments, the first measure of the importance is a qualitative measure. For example, the patient 192 may take a satisfaction assessment in which the patient 192 ranks how important the respective health-category is for them (e.g., a 1 is not important, and a 10 is a highly important).

In some embodiments, the developing 115 of the initial patient wellness assessment occurs during or after the first visit of the patient 192.

The patient 192 upon receiving the request to complete the wellness assessment, completes 120 the initial wellness assessment. In some embodiments, the completing 120 the assessment may include assigning scores to the one or more health-related categories. In some embodiments, the initial wellness assessment includes a ‘patient 192 notes’ region for the patient to extrapolate why they scored each question the way they did, or to add any other information. In some embodiments, the initial wellness assessment includes questions relating to the patient's 192 health in which they must answer or explain. For example, a series of questions relating to the patient's environment may ask the patient to describe their relationship with their family (e.g., spouse, parents, children, partner, etc.), their community, etc. In some embodiments, the patient completes the wellness assessment in person. In some embodiments, the patient receives the assessment via the server 196 and uploads their responses to the server 196.

In some embodiments, the patient completes 120 the initial wellness assessment during the first month of the program. For example, the patient completes “Initial Wellness Assessment” during the first month of the coaching program, as illustrated in Table 1.

In some embodiment, the coach 198 then reviews 125 the initial wellness assessment and the scores assigned to each health-related category. For example, during the “Initial Wellness Assessment” meeting during the first month of the program, the coach and the patient discuss the satisfaction scores, priority scale, health-related categories of focus, a health vision and a health coaching plan, as illustrated in Table 1. In some embodiments, the coach reviews 125 the initial wellness assessment with the patient 192 during a first epoch (e.g., the first visit) with the patient 192. In some embodiments, the coach 198 identifies or assists the patient to identify the health-related categories of concentration based on the wellness assessment. Included in reviewing 125 the wellness assessment, the coach 198 discusses various motivations and goals with the patient. In some embodiments, the motivations are one or more values that the patient 192 holds significant. In some embodiments, the motivations are one or more personal experiences that the patient 192 has had. For example, a patient 192 who has been called “big” by several people may use that experience as a motivation to lose weight. In some embodiments, the motivations are one or more behaviors of the patient 192. In some embodiments, the one or more motivations is an intrinsic motivation to accomplish a specific goal (e.g., a patient 192 desires to lose 20 lbs. in order to have more energy raising her children). In some embodiments, the one or more motivations is an extrinsic motivation to accomplish a specific goal (e.g., a company has a program for its employees in which every employee that reaches 10,000 steps a day for a week receives a t-shirt). In some embodiments, the one or more motivations may be a motivation to accomplish a specific goal relating to a health issue (e.g., a patient 192 has Type-2 diabetes and would like to lose 50 lbs. to decrease or reverse the effects of the disease).

In some embodiments, the coach 198 develops 127 a health vision plan for the patient 192. In some embodiments, the health vision plan is developed based on the completed 120 wellness assessment, reviewing 125 of the wellness assessment and discussing the motivation and the goals with the patient 192. In some embodiments, the patient 192 reviews the health vision plan developed 127 by the coach 198. In some embodiments, the patient 192 and coach 198 will create the health vision plan together. In some embodiments, once the health vision plan is created and sent to the patient 192 for review of the health vision plan, the patient may send back suggestions to change the health vision plan. In some embodiments, the suggestions are requested changes to the health vision plan. In some embodiments, creating the health vision plan is an iterative method in which the patient 192 and coach 198 make changes to the health vision plan back and forth until a plan is agreed upon. In alternate embodiments, the patient 192 reviews the health vision plan created by the coach 198 during the first month of treatment for the patient 192.

In some embodiments, developing 127 the health vision for patient 192 includes identifying the patient's desired health vision. The patient's desired health vision includes an understanding of how would the patient feel and look like when she is at her ideal level of wellness. For example, the patient may desire to fit into an old dress, run for a 10K race, sleep through the nights, or have new hobbies.

In some embodiments, developing 127 the health vision for patient 192 includes identifying the patient's life values. For example, the coach 198 may ask the patient 192 to identify the most important values of her life and how her health and wellness is connected with the most important values of her life.

In some embodiments, developing 127 the health vision for patient 192 includes identifying the best experiences the patient 198 has had in her life. For example, what have been the experiences where and when the patient 198 has felt alive and fully engaged and what experiences in general excite the patient 198 the most. Developing 127 the health vision for patient 192 includes also identifying how the best experiences the patient 198 has had in her life relate to her desired health vision.

In some embodiments, developing 127 the health vision for patient 192 includes identifying motivators that could drive the lifestyle changes of the patient 198. For example, the coach 198 requests the patient 198 to identify why the desired health vision is important for her, and what is the good that will come out of reaching the desired health vision.

In some embodiments, developing 127 the health vision for patient 192 includes identifying strategies that are effective for overcoming challenges during the program.

In some embodiments, developing 127 the health vision for patient 192 includes identifying a gap between the desired health vision and the patient's present wellness status (e.g., identifying an end point and a starting point). In some embodiments, identifying the gap between the desired health vision and the patient's present wellness status includes imagining a best case scenario for the success of the program and a worst case scenario for the success of the program.

In some embodiments, developing 127 the health vision for patient 192 includes identifying challenges and barriers that the patient is most concerned about with respect to achieving the desired health vision and identifying strengths that can be used to overcome such challenges and barriers. In some embodiments, developing 127 the health vision for patient 192 includes identifying people, resources, systems, and environments that support the patient to overcome such challenges and barriers.

In some embodiments, developing 127 the health vision for patient 192 includes identifying the patient's strengths. For example, is the patient creative, organized, compassionate, detail oriented, curious, critical thinker, passionate, determined, self-aware, empathetic, merciful, careful, playful, faithful, light-hearted, funny, and/or disciplined.

The coach 198 then sends a request for the patient 192 to define 130 one or more goals to achieve and one or more action steps that support achieving the one or more goals. In some embodiments, the one or more goals create a path between the subject's current health and the subject's desired health, as defined in the developed health vision plan 127.

In some embodiments, the coach 198 provides directions to identify the goals. In some embodiments, the coach 198 guides the patient 192 to identify S.M.A.R.T. goals. In some embodiments, the S.M.A.R.T. stands for specific, measureable, achievable, relevant, and time bound. A goal that is specific is significant and identifiable. For example, a broad goal might be to be a faster runner, a specific goal would be to break an 8 minute mile. A goal is measurable if it can be measured and tracked. For example, if a patient 192 wants to lose body fat, they may set a goal of losing 5% body fat, which can be measured and tracked on a body fat monitor (e.g., scale). An attainable goal is a goal that is feasible and/or reasonable to achieve, and the patient 192 is passionate and motivated to complete the goal. Additionally, the goal must be relevant to the patient 192 in that the goal is resourced, and results-based. Finally, the goal must be time bound. For example, the patient 192 may decide that they would like to lose 10 pounds in three months. In this example, three months is a defined time that the goal must be completed in. In some embodiments, the coach 198 walks the patient through identifying the S.M.A.R.T. goals that are specific to the patient 192.

In some embodiments, the goals are short-term goals (e.g., can be accomplished in <6 months). In some embodiments, the goals are long-term goals (e.g., can be accomplished in >6 months). In some embodiments, the long term goals are at least twice as long as the short term goals. In some embodiments, the goals include one or more long term goals and one or more short term goals. For example, throughout the progress of the program illustrated in Table 1, the patient has a long term (e.g., a 6 month goal) and reoccurring short term goals (e.g., 30 day goals). The goals are reviewed every 30 days during the months from two to six. For example, the 30 day goal is redefined once a month.

In some embodiments, in discussing motivations with the patient 192, the coach 198 identifies one or more strategies that will be effective for the patient 192 to achieve the patient's health goals. In some embodiments, the patient 192 identifies strategies that has been successful for them in the past, or that they believe will be successful for them currently. In some embodiments, the coach 198 provides suggested strategies that have been successful for other patients or that they believe will be successful for the patient 192.

In some embodiments, the coach 198 provides directions to identify one or more strategies the subject believes will be effective to achieve the one or more identified goals. The coach 198 further provides directions to identify, based on the one or more strategies, the one or more actions steps that support achieving the one or more identified goals. In some embodiments, the one or more action steps are associated with the long term and short term goals. For example, if the patient's goal is to lose a certain amount of weight during a short time period, the patient's action step to support this goal may include doing a 3 mile walk five times during the short time period. As another example, if the patient's goal is to increase the amount of vegetable eaten during a short time period, the patient's action step to support this goal may include eating a vegetarian meal once a day during the short time period. As illustrated in Table 1, the progress toward achieving the one or more short term and long term goals (e.g., 30-day and 6-month goals) and the associated action steps is done monthly during the second through sixth month of the program.

In some embodiments, the method 100 includes the server 196 storing 135 the goals. In some embodiments the server stores 135 blueprints and escalation points based on clinical decision-making algorithms. In some embodiments, the blueprint includes a plurality of information. In some embodiments, the blueprint includes a specific topic relating to a diagnosis. For example, a blueprint may include an overview of the diagnosis with a definition and description of a respective health condition, suggestions for goals and action steps that support managing the respective health condition, and resources and materials providing information about the respective health condition.

In some embodiments, the blueprint includes a list of potential lifestyle modifications relating to the diagnosis. In some embodiments, the blueprint includes a pre-defined list of questions. The list of questions may be a sample initial wellness assessment. In some embodiments, the blueprint includes an example for goals and action steps. In some embodiments, the goals and action steps includes a vision goal (e.g., ‘to improve my breathing, I will quit smoking by weaning myself off of nicotine. This will allow me to increase my stamina so that I can walk my daughter down the aisle at her wedding. To improve my quality of life and make breathing easier, I will consistently use my inhaler . . . ’). In some embodiments, the vision goal is created by the patient 192 as a short and personal motivator. In some embodiments, the blueprint comprises a statement for the health coach 198 on their responsibilities. The blueprint may include links (e.g., pod casts, TED talks, YouTube videos, articles, handouts, etc.) for educating patients and coaches.

In some embodiments, the escalations plans relating to the worsening of a condition (e.g., disease) or an emergency situation. In some embodiments, the escalation plan relates directly to a patient's condition. In some embodiments, the escalation plan includes symptoms relating to the patient's condition. In some embodiments, the escalation plan includes steps and suggestions for treatment. In some embodiments, the escalation plan includes a referral form for the health coach 198 to fill out and submit to another health professional. In some embodiments, the escalation plan includes a list of requirements for the patient 192 to get approved (e.g., by a physician) before working with a health coach 198.

In some embodiments, the server 196 stores 135 the goals, blueprints, and escalation plans during the first visit of the patient 192.

In some embodiments, the method 100 includes the coach 198 reviewing 140 the goals, action steps and recommendations with the patient 192 and reviewing any relevant blueprints and escalation plans with the patient 192. In addition to reviewing the goal and recommendation from the server 196, the coach 198 develops a plan to guide the patient 192. In some embodiments, the health vision plan is presented to the patient 192 in an app, in which the app comprises a list of activities for the patient 192 to accomplish. In some embodiments, the health vision plan is a physical list for the patient 192 to accomplish.

In some embodiments, reviewing 140 the goals, action steps and recommendations with the patient 192 includes receiving an approval from the physician 194 that the subject may exercise. In some embodiments, the coach 198 determines, based on the respective preexisting health condition and whether the patient 192 has experienced any symptoms of a plurality of symptoms during exercising, whether the patient 192 has any limitations on ability to exercise. In some embodiments, the coach 198 educates the patient 192 on a type and amount of exercise that would support the management of the health condition, and facilitating definition of at least one action step for the patient 192 to take in furtherance of achieving the long or short term goal.

In some embodiments, the coach 198 reviews 140 the goals and recommendations during the first month of the program. In alternate embodiments, the coach 198 reviews 140 the goals and recommendations and develops the health vision plan during the first month of treatment for the patient 192.

In some embodiments, once the health vision plan is agreed upon, the coach 198 confirms 150 the health vision plan and sends a request for the patient 192 to input (e.g., upload into server 196) periodic health data. In some embodiments, periodic health data includes body measurements or pictures, data relating to the patient's condition (e.g., heart rate, blood sugar levels, blood pressure, etc.), or any reasonable health data thereof. In some embodiments, the server 196 stores the periodic health data 155 and allows the data to be accessed by the coach 198 and the patient 192. In some embodiments, periodic health data is uploaded on a schedule (e.g., biweekly, bimonthly, etc.) in order to track patient progress.

In some embodiments, the coach 198 confirms 150 the health vision plan and sends a request for the patient 192 to input periodic health data during the first visit of the patient 192. In alternate embodiments, the coach 198 confirms 150 the health vision plan and sends a request for the patient 192 to input periodic health data during the first month of treatment for the patient 192.

In some embodiments, the coach 198 approves the recommended updates and send a request to the patient 192 to upload 160 more periodic health data during the month of the program. In some embodiments, the coach 198 approves the recommended updates and send a request to the patient 192 to upload 160 more periodic health data during the following months of treatment.

In some embodiments, upon accessing the periodic health data, the coach 198 provides 165 notes and/or escalation suggestions into the server 196. In some embodiments, the notes relate to patient 192 appointments. In some embodiments, the notes relate to patient progress (e.g., patient chart information). In some embodiments, the escalation suggestions are a change to the patient's program. In some embodiments, the escalation suggestions are more difficult tasks for the patient 192 to perform. In some embodiments, the escalation suggestions are symptoms for the patient 192 to track.

In some embodiments, the coach 198 provides 165 notes and escalation suggestions during the first month of treatment for the patient 192. In some embodiments, the coach 198 uploads the notes and escalation suggestions to the server 196 (e.g., to be reviewed by the physician 194). In some embodiments, the coach 198 provides the notes and escalation suggestions to the patient 192. In some embodiments, the escalating includes contacting the patient 198, e.g., electronically, to alert them of recommendations for addressing the preexisting health condition.

In some embodiments, the server 196 stores the notes 170 and escalation suggestions and provides recommended updates to the health vision plan. In some embodiments, the coach 198 approves the recommended updates and sends the updated plan to the patient 192.

In some embodiments, the server 196 stores the notes 170 and provides recommended updates to the health vision plan during the first month of treatment for the patient 192.

In some embodiments, the physician 194 accesses the stored notes 170 on the server for review and evaluation. In some embodiments, the physician 194 may access the stored notes 170 at any time during the program in order to monitor the patient's progress and health. In some embodiments, the physician 172 provides recommendations and/or treatment guidance. The recommendations may be provided, via the server 196 or directly, to the coach 198 and/or the patient 192.

In some embodiments, the coach 198 rejects the recommendations. In some embodiments, after the health vision plan is updated, the patient 192 is sent a program and wellness satisfaction survey to complete. In some embodiments, the patient 192 completes 175 a program and wellness satisfaction survey at regular intervals (e.g., monthly). In some embodiments, the program and wellness satisfaction survey is a questionnaire for the patient 192 to fill out, which provides feedback for the coach 198 based on the program or the patient's own wellness. In some embodiments, a lower wellness satisfaction survey score will require a program modification.

In some embodiments, the patient 192 completes 175 a program and wellness satisfaction survey at regular intervals during the first month of treatment for the patient 192. In some embodiments, the patient 192 completes 175 a program and wellness satisfaction survey at regular intervals during the following months of treatment.

In some embodiments, the coach 198 performs 180 coaching sessions, evaluates wellness satisfaction scores, and reviews goals with the patient 192. For example, through the second to sixth month of the program the follow up visits include reviewing wellness satisfactions scores, reviewing program satisfaction surveys, and reviewing status of 30-day and 6-month goals, as illustrated in Table 1. In some embodiments, the coaching session relates to the patient's wellness plan (e.g., personal training, physical therapy, psychiatry, etc.). In some embodiments, the coaching session is conducted as a teleconference or a videoconference. In some embodiments, the coaching session is conducted online.

In some embodiments, the coach 198 performs 180 coaching sessions, evaluates wellness satisfaction scores, and reviews goals with the patient 192 during the first month of treatment for the patient 192. In some embodiments, the coach 198 performs 180 coaching sessions, evaluates wellness satisfaction scores, and reviews goals with the patient 192 during the following months of treatment.

In some embodiments, the coach 198 transmits messages 185 to the patient. In some embodiments, transmitting the messages 185 is performed through the server 196 (e.g., via a mobile app). In some embodiments, the messages are transmitted to the patient directly (e.g., as text messages or e-mails). In some embodiments, the messages are sent regularly (e.g., daily, weekly, at a predefined time). In some embodiments, the messages are in form of text messages, phone calls, video appointments, mail, or any reasonable messaging system thereof. In some embodiments, the messages may be words of encouragement, reminders, or information relating to a health category. Similarly, the patient 192 transmits messages to the coach through the server 196 or directly.

In some embodiments, the coach 198 transmits messages 185 through the server 196 to the patient during the first month of treatment for the patient 192. In some embodiments, the coach 198 transmits messages 185 through the server 196 to the patient during the following months of treatment.

In some embodiments, the coach 198 meets with the physician 194 and the patient 192 for milestone meetings 190. For example, the coach meets with the patient for milestone visits once a month, as illustrated in Table 1. The milestone meetings 190 may be an evaluation of the patient's condition. In some embodiments, the physician 194 performs tests to evaluate the patient's condition. In some embodiments, the patient 192 re-evaluates their goals (e.g., 30-days and 6-month goals in Table 1). In some embodiments, the patient 192 creates a new set of goals.

In some embodiments, the coach meets with the physician 194 and the patient 192 for milestone meetings 190 during the first month of treatment for the patient 192. In some embodiments, the coach 198 meets with the physician 194 and the patient 192 for milestone meetings 190 during the following months of treatment.

Method 100 described with respect to FIG. 1 has been demonstrated to provide for an effective telemedicine procedure for management of a patient's health condition. As shown in a recent study on patients diagnosed with obesity, at 12 weeks an adherence of 92% was found in patients participating in the described telemedicine coaching program (e.g., method 100) whereas only 75% adherence was found in patients not participating in the program but were instructed to follow the same recommended diet program. Alencar et al., “Telehealth-Based Health Coaching Increases m-Health Device Adherence and Rate of Weight Loss inObese Participants,” Telemedicine and e-Health, DOI 0.1089/tmj.2019.0017 (2019). The results demonstrate that method 100 described above provides for an improved and effective telemedicine method for managing a health condition in a subject (e.g., patient) by reinforcing lifestyle adjustment.

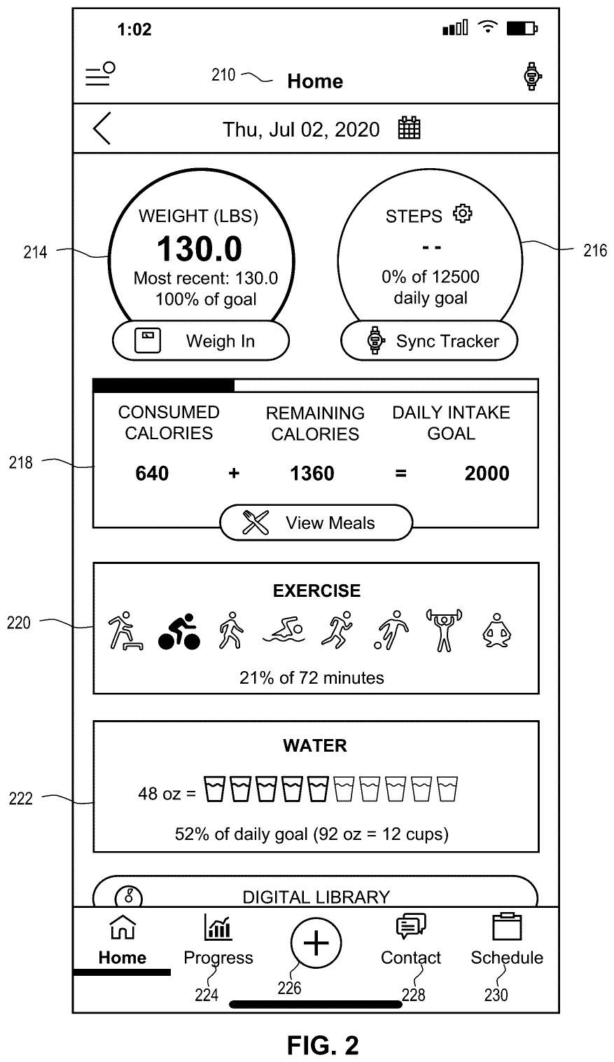

FIGS. 2-10 illustrate an example of a graphical user interface (GUI) on the device of a user. In some embodiments, the GUI displays a mobile application associated with the server 196 in FIG. 1. The mobile application enables interactions between the coach 198 and the patient 192. In some embodiments, the interactions include uploading periodic health data 160, completing wellness assessments 120, completing program and wellness satisfaction surveys 175, transmitting messages 185, and/or any other interactions between the coach 198 and the patient 192.

In some embodiments, the GUI is presented to the patient 192 by use of a server 196. In some embodiments, the GUI is a software application. In some embodiments, the GUI contains a plurality of windows, wherein each window corresponds to a health plan. In some embodiments, the plurality of windows includes a window for each of the health categories. The GUI may include a home screen 210 that displays a plurality of information relating to the patient 192. For example, the home screen 210 may include a calendar tab 212, wherein the patient 192 is able to select a specific date to view and edit.

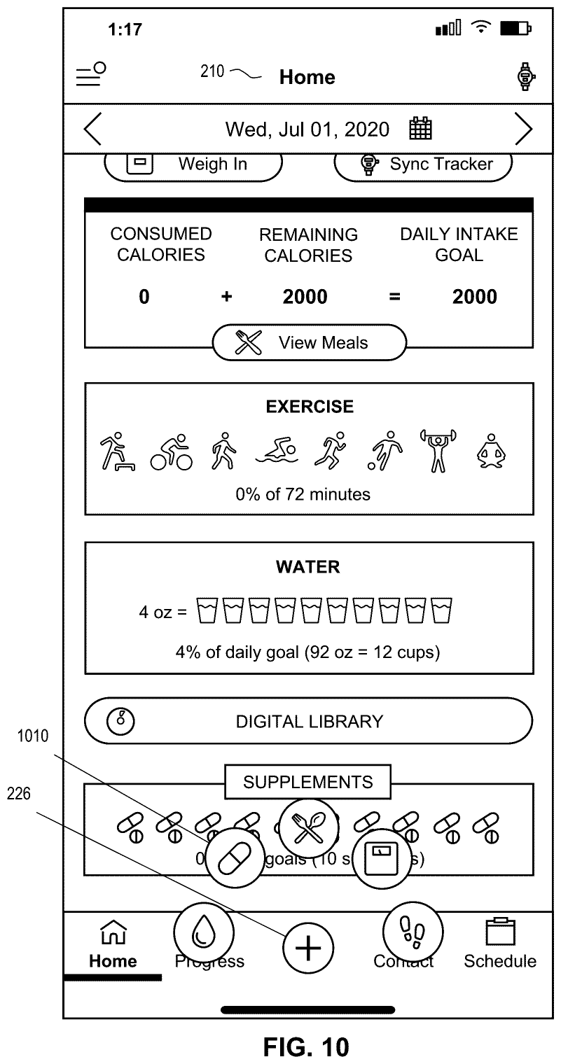

In some embodiments, the home screen 210 displays an overview of a weight tracker 214, as shown in FIG. 2. The weight tracker displays the most recent recorded weight of the patient 192. In some embodiments, the weight tracker may include a percentage of weight lost in order for the patient to reach their goal weight. For example, if the patient 192 desires to lose 20 lbs. and the user has already lost 10 lbs., the percentage would show 50% of goal weight. In some embodiments, the patient 192 has the ability to navigate to another screen (also shown in FIG. 10) in order to update their weight.

In some embodiments, the home screen 210 displays an overview of a step tracker 216. In some embodiments, the step information is automatically updated by pairing the software application with a patient's 192 phone. In some embodiments, the step information is automatically updated when the software application is paired with an accelerometer (e.g., fitbit). In some embodiments, the patient 192 manually inputs their step information. In some embodiments, the step tracker 216 includes displaying a step goal and the patient's 192 percentage reached toward the goal. For example, if the patient 192 has a goal of taking 12,500 steps in a day, a step count of 7,500 would show as 60% of the daily goal.

In some embodiments, the home screen 210 displays an overview of a nutrition tracker 218. In some embodiments, the nutrition tracker includes a consumed calories count, a remaining calories count, and a daily intake goal. In some embodiments, the nutrition tracker 218 only displays how many calories left the patient 192 has for that day, before reaching their calorie limit. In some embodiments, the software application can pair with another software application to update the nutrition tracker 218 to show calorie information.

In some embodiments, the home screen 210 displays an overview of an exercise tracker 220 that the patient 192 has uploaded. In some embodiments, the exercise tracker 220 includes information relating to the patient's 192 activity. For example, the patient 192 is able to update the software application to reflect the various physical activities (bike riding, swimming, running, playing soccer, lifting weights, running, yoga, etc.) that the patient 192 has performed in a day. In some embodiments, the exercise tracker 220 keeps track of the amount of minutes that a patient 192 has performed a specific task. The exercise tracker 220 may keep track of the amount of minutes by including a stopwatch that the patient 192 can start and stop. In another embodiment, the exercise tracker 220 may update the exercise when the software application is linked to an accelerometer (e.g., fitbit, exercise tracker, application, etc.). In some embodiments, the exercise tracker 220 includes a percentage of completion based on the amount of time that the user has performed the activity (e.g., 21% of 72 minutes completed).

In some embodiments, the home screen 210 displays an overview of a water tracker 222. In some embodiments, the water tracker 222 displays an amount of water that the patient has consumed. In some embodiments, the user updates the water tracker 222. In some embodiments, the water tracker 222 includes a percentage of completion of a water intake goal. For example, the water tracker might show that 52% of the patient's 192 daily water intake has been met (e.g., for an intake of 92 fl. oz. or 12 cups).

In some embodiments, the home screen 210 displays a plurality of navigation tabs (e.g., progress tab 224, navigation tag 226, contact tab 228, schedule tag 230, etc.). In some embodiments, the patient 192 navigates to one or more tags to view their progress (e.g., progress 224), upload information relating to their program (e.g., navigation tag 226, upload information relating to calories consumed, activity, water intake, etc.), contact their health coach 198, or a physician 194 (e.g., contact tab 228), or view their training and/or appointment schedules (e.g., schedule tab 230).

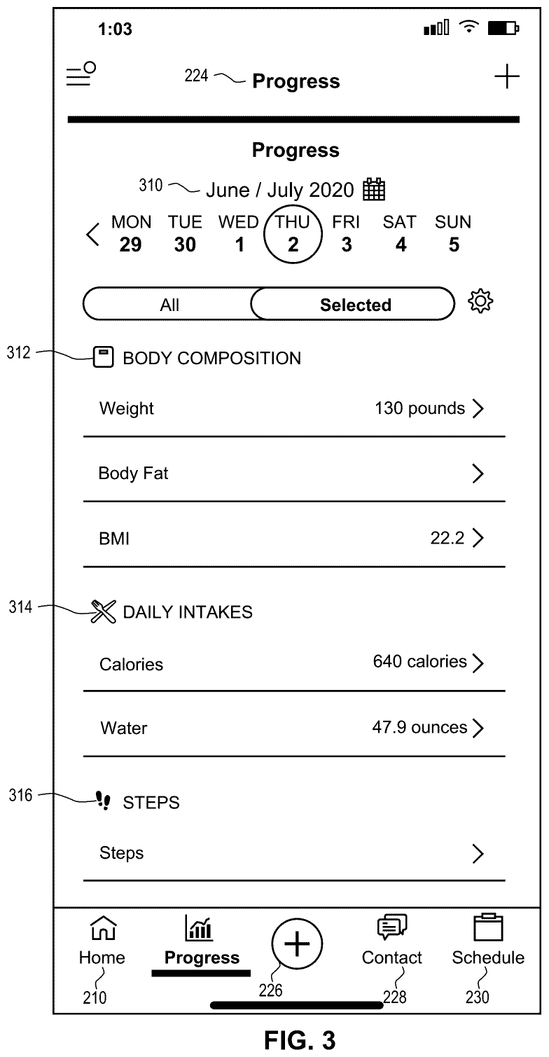

FIG. 3 illustrates a progress tracking feature on a graphical user interface as introduced in FIG. 2. The progress tab 224 includes a plurality of information relating to the patient 192 and their progress through the program. The progress tab 224 includes calendar information 310 for the patient to view their progress on a specific day or over the course of several days. In some embodiments, the progress tab 224 includes a body composition region 312. The body composition region 312 includes a plurality of information relating to the patient's 192 body composition. In some embodiments, the body composition region 312 includes the patient's 192 weight information. In some embodiments, the body composition region 312 includes the patient's 192 body mass index percentage. In some embodiments, the body composition region 312 includes the patient's 192 body measurements (e.g., height, left bicep diameter, right bicep diameter, waist length, chest length, etc.). In some embodiments, the body composition region 312 is updated by the patient 192. In some embodiments, the body composition region 312 is updated when the software application is linked with another Bluetooth device (e.g., scale).

In some embodiments, the progress tab 224 includes a daily intake region 314. In some embodiments, the daily intake region 314 tracks daily intake of the patient 192 from a plurality of sources. In some embodiments, the daily intake region 314 includes tracking the patient's 192 caloric consumption. In some embodiments, the daily intake region 314 includes tracking the patient's 192 water intake. In some embodiments, the daily intake region 314 includes tracking the patient's 192 vitamin and/or supplement intake. In some embodiments, the daily intake region 314 includes tracking the patient's 192 medication intake.

In some embodiments, the progress tab 224 includes a step counting region 316. In some embodiments, the step counting region 316 displays the patient's 192 number of steps per day.

In any of the above embodiments in the progress tab 224, the user may select a date range and a specific tracking region (e.g., body composition region 312, daily intake region 314, step counting region 316, etc.) and generate a graph that displays the patient's 192 progress over time. In some embodiments, the progress tab 224 automatically displays a chart showing the patient's 192 progress.

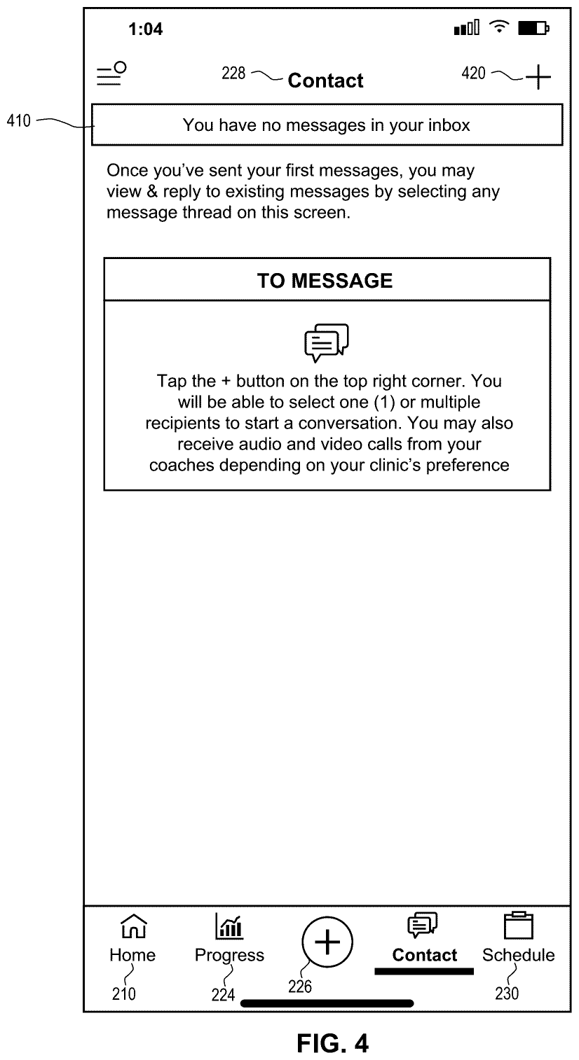

FIG. 4 illustrates a messaging feature on a graphical user interface. In some embodiments, the messaging feature is contact tab 228. The contact tab 228 displays messages to the user from the coach 198, a physician 194, other program members, etc. In some embodiments, the contact tab 228 includes a summary 410 which gives information on unread or recent messages sent and received by the patient 192. The summary 410 may include messages that the patient 192 can respond directly to. In some embodiments, the contact tab 228 includes a button 420 for the patient 192 to compose a new message. In some embodiments, the button 420 can also send audio and video calls to the patient's 192 coach 198 or physician 194.

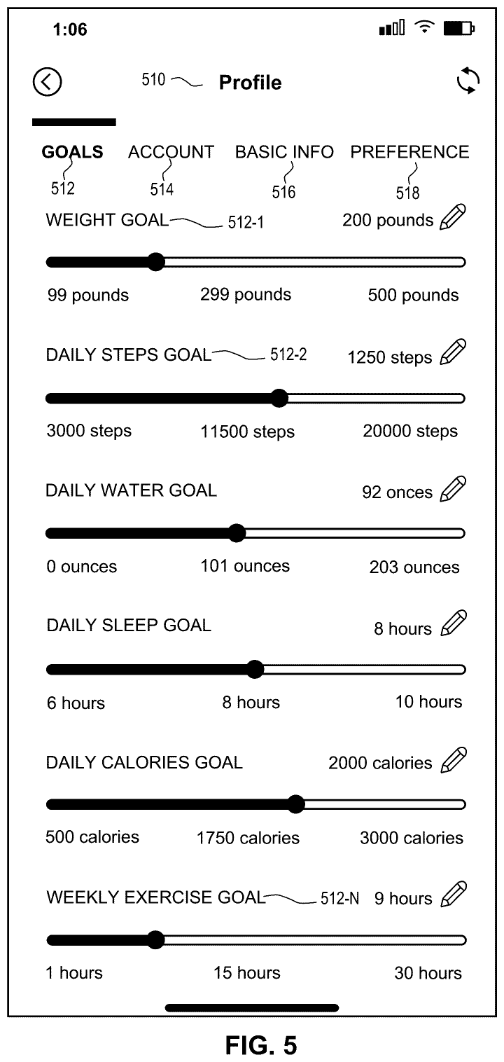

FIG. 5 illustrates a profile feature on a graphical user interface. In some embodiments, a profile tab 510 includes a plurality of information relating to the patient 192. In some embodiments, the profile tab 510 includes a plurality of sub-tabs. In some embodiments, the profile tab 510 includes a goals sub-tab 512 which lists the patient's 192 identified goals (e.g., a first goal 512-1, a second goal 512-2, and goal N 512-N). In some embodiments, the goals sub-tab 512 includes a first goal 512-1 that shows the patient's 192 progress on the first goal 512-1. The showing of the patient 192 progress may be in a percentage, chart, etc. For example, a first goal 512-1 might be a specific weight goal. In this example, the patient's 192 progress may be shown based on how much weight they have lost on a bar chart.

In some embodiments, the goals sub-tab 512 includes a second goal 512-2 that shows the patient's progress on the second goal 512-2. In some embodiments, the first goal 512-1, or the second goal 512-2 may be filtered based on a short term goal (e.g., minimum number of steps in a day), or a long term goal (e.g., weight loss over a month). In some embodiments, the profile tab 510 includes an account sub-tab 514 which includes information relating to the patient's 192 subscription information. In some embodiments, the profile tab 510 includes a basic information sub-tab 516. The basic information sub-tab may include patient information (e.g., name, email, phone number, coach 198, physician information, hospital information, emergency contact, address, height, age, sex, etc.). In some embodiments, the profile tab 510 includes a preference sub-tab 518 which includes information relating to the patient's 192 preferences.

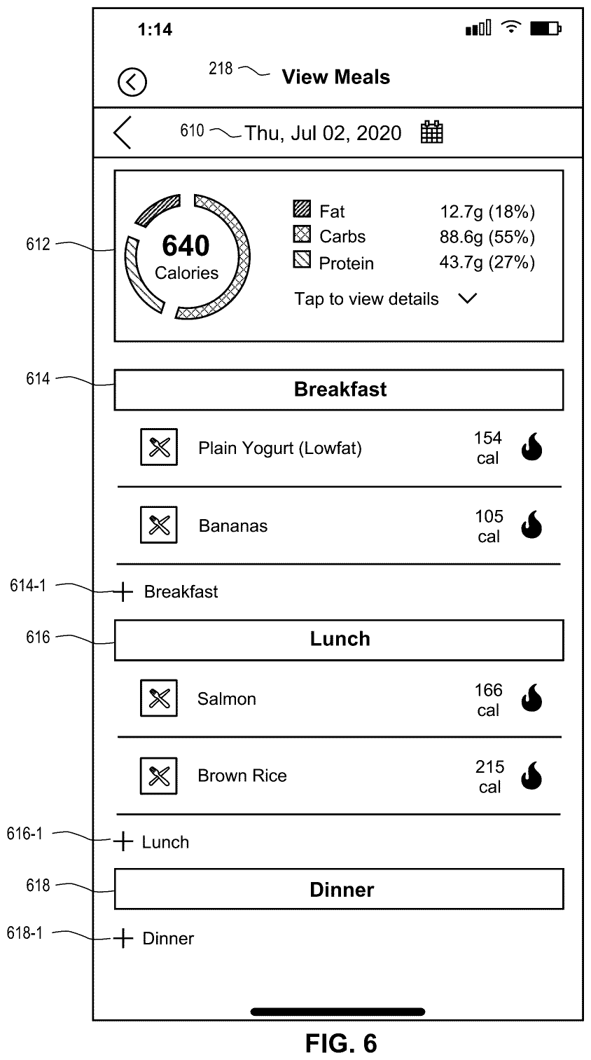

FIG. 6 illustrates a nutrition tracking feature on a graphical user interface. In some embodiments, the nutrition tracking feature is nutrition tracker 218. In some embodiments, nutrition tracker 218 tracks the patient's 192 meals and calories. In some embodiments, the nutrition tracker 218 includes a new nutrition tracking window for each day 610. In some embodiments, the nutrition tracker 218 includes a nutrition window 612. In some embodiments, the nutrition window 612 includes tracking the amount of calories that the patient 192 consumes daily. In some embodiments, the nutrition window 612 includes a breakdown of the types of nutrition (e.g., fat, carbs, protein, etc.) consumed each day. For example, a patient 192 has consumed 640 calories in a single meal. In this example, the breakdown of the calories may include 12.7 g (18%) fat, 88.6 g (55%) carbs, and 43.7 g (27%) protein. In this example, the breakdown can be used for the coach 198 or the physician 194 to understand the patient's 192 progress or lack thereof. In some examples, the nutrition window includes an entire breakdown of each food consumed (e.g., vitamins, sugars, fats, sodium, etc.).

In some embodiments, the nutrition tracker 218 includes a breakfast window 614, a lunch window 616, a dinner window 618, and a snack window (not pictured). In each window, the patient 192 is able to upload food that the patient 192 consumed. In some embodiments, the breakfast window 614 includes a breakfast addition button 614-1 for the user to upload more foods into the breakfast window 614. In some embodiments, the lunch window 616 includes a lunch addition button 616-1 for the user to upload more foods into the lunch window 616. In some embodiments, the dinner window 618 includes a dinner addition button 618-1 for the user to upload more foods into the dinner window 618. In some embodiments, each addition automatically updates the nutrition window 612 to reflect the newly added foods. In some embodiments, each food addition includes a calorie count associated with the food. In some embodiments, the patient 192 can edit the serving size and/or amount. In some embodiments, the patient 192 can update the calorie count for each food.

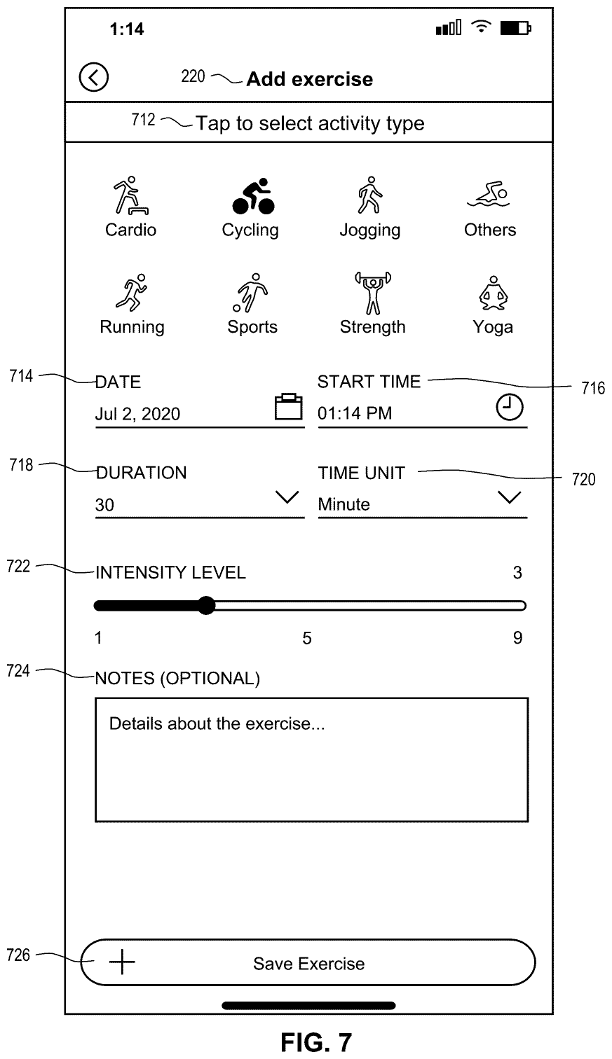

FIG. 7 illustrates a fitness tracking feature on a graphical user interface. In some embodiments, the fitness tracking feature is exercise tracker 220. In some embodiments, exercise tracker 220 includes an activity type region 712 for the patient 192 to select. In some embodiments, the activity type region 712 includes a plurality of physical activities and exercises (e.g., cardio, cycling, jogging, swimming, running, sports, strength, yoga, stretching, etc.). In some embodiments, the user makes a selection and includes information relating to the selection. In some embodiments, the exercise or activity selection includes a required date entry 714. In some embodiments, the date entry 714 automatically updates to the current date. In some embodiments, the exercise tracker 220 includes a start time entry 716. In some embodiments, instead of a start time entry 716, the patient 192 can start the stop watch (not pictured). In some embodiments, the start time entry 716 is updated from another software program or fitness tracker (e.g., health data, fitbit, etc.). In some embodiments, the exercise tracker 220 includes an end time entry (not pictured).

In some embodiments, the exercise tracker 220 includes a durational time entry 718. In some embodiments, the exercise tracker 220 includes a time unit entry 710. In some embodiments, the time unit is selected from a drop down menu comprising milliseconds, seconds, minutes, hours, days, weeks, months, years, or a combination thereof.

In some embodiments, the exercise tracker 220 includes an intensity level 722, wherein the patient 192 may include the intensity of the exercise. In some embodiments, the intensity level 722 directly correlates to the patient's 192 heart rate. In other embodiments, the intensity level 722 directly correlates to how difficult the patient 192 found the exercise. In some embodiments, the intensity level ranges from 1-10, wherein 1 represents an easy intensity and 10 represents an extremely difficult intensity. In some embodiments, the intensity level ranges from 1-5, wherein 1 represents an easy intensity and 5 represents an extremely difficult intensity. In some embodiments, the intensity level 722 is automatically updated based on patient 192 heart rate. In other embodiments, the patient 192 manually inputs the intensity.

In some embodiments, the exercise tracker 220 includes an optional notes section 724. In some embodiments, the patient 192 can update the optional notes section 724 with information about the exercise (e.g., difficulty, heart rate, soreness, etc.).

In some embodiments, the exercise tracker 220 includes a save exercise feature 726 that saves the information relating to the exercise.

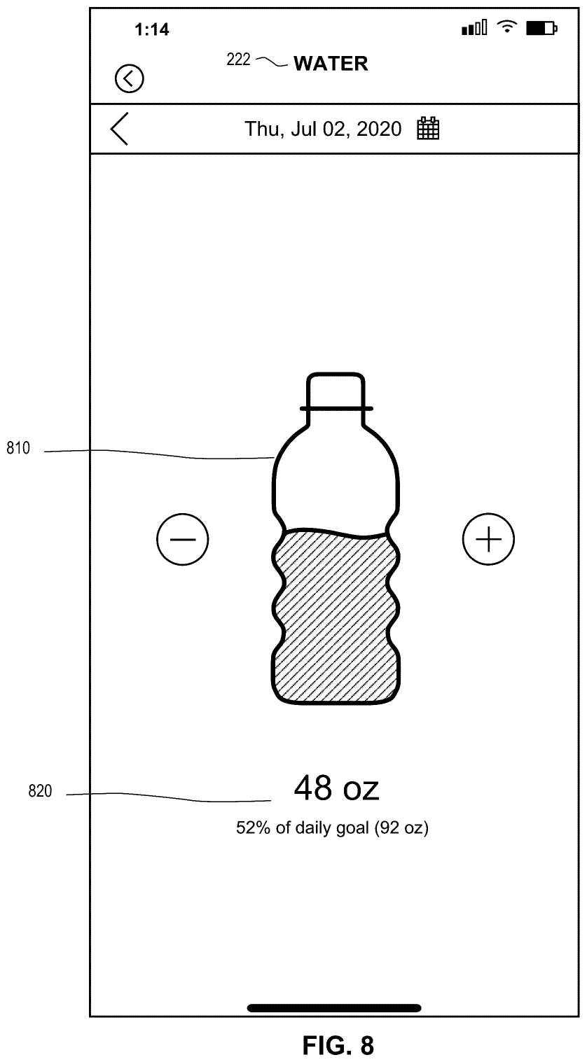

FIG. 8 illustrates a water intake feature on a graphical user interface. In some embodiments, the eater intake feature is water tracker 222. In some embodiments, water tracker 222 includes an illustration of a water bottle 810. In some embodiments, the illustration is an outline of a water bottle. In some embodiments, the water bottle 810 changes pixel color or darkens in color once the user has uploaded a water intake amount. In some embodiments, the change in pixel color gives the illusion of the water bottle 810 filling. The water tracker 222 may also include an amount of water intake 820 (e.g., 48 oz.). In some embodiments, the water intake 820 includes the amount of water consumed towards the daily goal (e.g., 52% of daily goal).

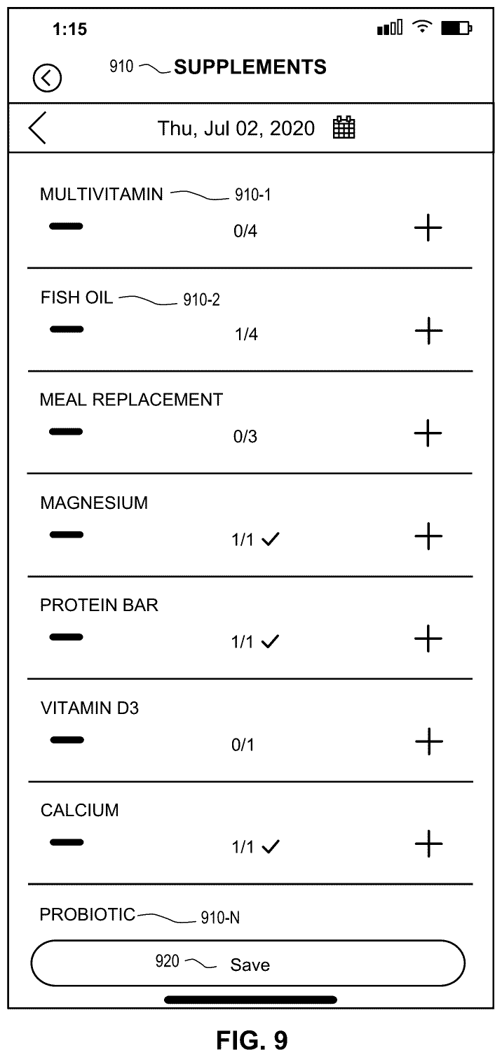

FIG. 9 illustrates a supplement tracking feature on a graphical user interface. The supplement tracker 910 includes tracking a plurality of supplements prescribed to a patient 192 by a physician 194 and/or health coach 198. In some embodiments, the supplement tracker 910 tracks a patient's 192 medication. In some embodiments, the supplement tracker 910 tracks a patient's 192 vitamins and nutrients. In some embodiments, the supplement tracker includes a first supplement (e.g., first supplement 910-1), a second supplement (e.g., second supplement 910-2), or a plurality of supplements (e.g., first supplement 910-1, second supplement 910-2, supplement N 910-N). In some embodiments, the plurality of supplements are provided to the user in a list. In some embodiments, when the patient 192 records that they have taken the supplement, the supplement is removed from the list. In some embodiments, the supplement tracker 910 includes a save feature 920, in which the patient 192 is able to save their recorded supplements.

FIG. 10 illustrates a navigation feature on a graphical user interface. In some embodiments, the navigation feature is the navigation tag 226. In some embodiments, the navigation tag 226 includes a pop-up menu 1010. In some embodiments, the pop-up menu 1010 is overlaid on top of the home screen 210. In some embodiments, the pop-up menu 1010 is a separate window. In some embodiments, the pop-up menu 1010 includes a plurality of navigation tools (e.g., water tracker 222, supplement tracker 910, nutrition tracker 218, weight tracker 214, step tracker 216, etc.). In some embodiments, upon selecting a navigation tool, the patient 192 is navigated to a new window to view and upload information relating to the specific navigation tool.

Various examples of aspects of the disclosure are described herein below. These are provided as examples and do not limit the subject technology. Identification of the figures and reference numbers are provided below merely as examples for illustrative purposes, and the clauses are not limited by those identifications.

In accordance with some embodiments, a method (e.g., method 100 described with respect to FIG. 1) for managing a health condition in a subject in need thereof is performed by reinforced lifestyle adjustment based on one or more underlying motivations. The method includes obtaining, from the subject at a first time point (e.g., occurring before the start of a first epoch and a second epoch), a first scored self-assessment (e.g., complete wellness assessment 120) for a plurality of health-related categories across a plurality of metrics. The first self-assessment includes, for each respective health-related category in the plurality of health-related categories, a first (e.g., quantitative) measure of the subject's assessment of their own health relative to the respective health-related category, a first (e.g., quantitative) measure of the subject's satisfaction with their own health relative to the respective health-related category, and a first (e.g., quantitative) measure of the subject's desire to improve their own health relative to the respective health-related category. The method includes selecting a first subset of health-related categories (i.e., less than all; e.g., from 1 to 5, from 1 to 4, from 1 to 3, 1 or 2, or 1 of the health-related categories) from the plurality of health-related categories based on the first scored self-assessment. The method includes facilitating subject creation of a health vision plan (e.g., develop health vision plan 127) for improving the health of the subject with respect to the selected subset of health-related categories using first information arising from one or more (e.g., telecommunication) meetings with the subject (e.g., “Initial Wellness Assessment” meeting in Table 1). The first information includes a description of the subject's desired health outcome elicited from the subject, and one or more values, one or more personal experiences, one or more behaviors, or one or more motivations of the subject elicited from discussion with the subject The method includes establishing a short term goal (e.g., a 30-day goal illustrated in Table 1) for improving the health of the subject (e.g., define goals and actions steps 130), relative to the selected subset of health-related categories, over a first epoch (e.g., 30 days or a month). The method includes establishing a long term goal (e.g., a 6-month goal illustrated in Table 1) for improving the health of the subject, relative to the selected subset of health-related categories, over a second epoch. The second epoch subsumes the first epoch and is at least twice as long as the first epoch. The short term goal and the long term goal create a path between the subject's current health and the subject's desired health with respect to the selected subset of health-related categories. For example, the path between the subject's current health and the desired health is identified by discussing starting and ending points, discussing best and worst case scenarios, identifying perceived obstacles/barriers to change, identifying the subject's support network, and identifying perceived strengths of the subject. The method includes identifying one or more strategies the subject believes will be effective to achieve the short or long term goals. The method includes identifying., based on at least the one or more identified strategies, at least one action step (e.g., define goals and action steps 130) for the subject to take in furtherance of achieving the short or long term goal. The method includes re-evaluating (e.g., evaluate wellness satisfaction scores and re-evaluate goals 180) the at least one action step using second information obtained from a primary follow-up (e.g., telecommunication) meeting with the subject. The primary follow-up meeting occurs after one or more initial meetings and during the first and second epochs (e.g., “Follow up” visit during the first month in Table 1). The second information includes a description of the subject's perception of their execution of the at least one action step elicited from the subject, and a determination of whether the subject wishes to modify the long term goal, the short term goal, or the at least one action step. When it is determined that the subject wishes to modify the long term goal, the short term goal, or the at least one action step, the long term goal, the short term goal, or the at least one action step is reestablished in modified form. The method includes re-evaluating the at least one action step during each of a series of secondary follow-up (e.g., telecommunication) meetings with the subject (e.g., “Follow up (Milestone) Visits” during months 2-6 in Table 1), where the series of secondary follow-up (e.g., telecommunication) meetings occur after the primary follow-up meeting and during the second epoch. The method also includes providing to the subject, outside of a meeting with the subject, one or more messages (e.g., electronic messages) (e.g., transmit messages 185) that include content configured to (i) encourage the subject to achieve the long term goal or the short term goal, (ii) remind the subject of one or more of the at least one action step, or (iii) educate the subject on the selected subset of health-related categories.

In some embodiments, the method further includes obtaining, from the subject at a second time point occurring after the first time point (e.g., at or near the end of the second epoch), a second scored self-assessment (e.g., complete wellness satisfaction surveys 175) for the plurality of health-related categories across the plurality of metrics, the second scored self-assessment including, for each respective health-related category in the plurality of health-related categories a first (e.g., quantitative) measure of the subject's assessment of their own health relative to the respective health-related category, a second (e.g., quantitative) measure of the subject's satisfaction with their own health relative to the respective health-related category, and a second (e.g., quantitative) measure of the subject's desire to improve their own health relative to the respective health-related category; and selecting a second subset of health-related categories from the plurality of health-related categories based on the second scored self-assessment.

In some embodiments, the method further includes monitoring one or more health parameters of the subject (e.g., the subject's blood pressure, weight, step count, blood glucose level, food intake) (e.g., periodic health data 160), the monitoring including receiving, at a first frequency (e.g., during the first epoch and the second epoch), records of the one or more health parameters of the subject at an electronic device (see, e.g., FIGS. 2-10), and reviewing the records received for the one or more health parameters. In some embodiments, the records are input manually by the subject, e.g., via a food journal. In some embodiments, the records are automatically generated upon measurement by a remote health monitoring device, e.g., a smart watch, scale, glucose monitor, pedometer, implanted cardiac monitor, etc.

In some embodiments, the monitoring the one or more health parameters includes monitoring the subject's blood pressure, weight, step count, movement, heart rate, blood glucose level, blood cholesterol, food intake, drink intake, and/or notes regarding the subject's emotional state (see, e.g., FIGS. 2-10).

In some embodiments, the electronic device is a health monitoring device and the records are automatically generated upon a measurement performed by the health monitoring device.

In some embodiments, the records are input manually by the subject on a client electronic device.

In some embodiments, the records include an amount or type of food or drink the subject has consumed within a period of time.

In some embodiments, the method further includes, prior to a respective secondary follow-up meeting in the series of secondary follow-up meetings, determining whether the monitored one or more health parameters indicate that the subject is taking the at least one action step in furtherance of achieving the short or long term goal (e.g., re-evaluate goals 180, 190); and while re-evaluating the at least one action step during the respective secondary follow-up meeting: when the monitored one or more health parameters indicate that the subject is taking the at least one action step in furtherance of achieving the short or long term goal, discuss with the subject (i) why the subject has been successful in taking the at least one action step, and (ii) whether to adjust the at least one action step to further support achievement of the short or long term goal; and when the monitored one or more health parameters indicate that the subject is not taking the at least one action step in furtherance of achieving the short or long term goal, discuss with the subject (i) why the subject may not be taking the at least one action step, and (ii) whether to adjust the at least one action step to further encourage the subject to take the at least one action step.

In some embodiments, the method further includes, prior to a respective secondary follow-up meeting in the series of secondary follow-up meetings, determining whether the monitored one or more health parameters indicate that the subject is progressing towards achievement of the short term goal or the long term goal (e.g., re-evaluate goals 180, 190); and while re-evaluating the at least one action step during the respective secondary follow-up meeting: when the monitored one or more health parameters indicate that the subject is progressing towards achievement of the short term goal or the long term goal, discuss with the subject (i) why the subject has been successful in progressing towards achievement of the short term goal or the long term goal, and (ii) whether to adjust the short term goal or long term goal to further improve the health of the subject; and when the monitored one or more health parameters indicate that the subject is not progressing towards achievement of the short term goal or the long term goal, discuss with the subject (i) why the subject may not be progressing towards achievement of the short term goal or the long term goal, and (ii) whether to adjust the at least one action step in furtherance of achieving the short term goal or the long term goal.

In some embodiments, the description of the subject's perception of their execution of the at least one action step elicited from the subject comprises: a description of a positive event that has occurred with respect to the subject's health condition since a previous meeting, and an identification of a strength that facilitated the positive event; and the determination of whether the subject wishes to modify the long term goal, the short term goal, or the at least one action step is based on at least the subject's perception of their execution of the at least one action step.

In some embodiments, the content of the one or more messages provided to the subject outside of a meeting with the subject is based on at least the review of the records received for the one or more health parameters.

In some embodiments, at least one message of the one or more messages provided to the subject outside of a meeting with the subject is automated and is provided at a predetermined time.

In some embodiments, the method further includes, when the subject has been diagnosed with a respective preexisting health condition in a plurality of preexisting health conditions, determining, based on the reviewing the records received for the one or more health parameters, whether the subject has experienced a triggering event associated with the respective preexisting health condition. When it is determined that the subject has experienced the triggering event, escalating the subject's health condition to a medical professional according to an escalation plan corresponding to the preexisting health condition (e.g., provide notes and escalations suggestions 165). In some embodiments, the escalating includes contacting the subject, e.g., electronically, to alert them of recommendations for addressing the preexisting health condition. In some embodiments, the escalating includes contacting a medical professional, e.g., electronically, to alert them of the subject's health condition.

In some embodiments, the escalating includes contacting the subject and providing the subject recommendation for addressing the triggering event associated with the respective preexisting health condition.

In some embodiments, the method further includes, when the subject has been diagnosed with a respective preexisting health condition in a plurality of preexisting health conditions, during a respective secondary follow-up meeting in the series of follow-up meetings, determining whether the subject has experienced one or more symptom of a triggering event associated with the respective preexisting health condition. When it is determined that the subject has experienced the triggering event, escalating the subject's health condition to a medical professional (e.g., provide notes and escalation suggestions 165) according to an escalation plan (e.g., recommend escalation plans 135) corresponding to the preexisting health condition. In some embodiments, the escalating includes contacting the subject, e.g., electronically, to alert them of recommendations for addressing the preexisting health condition. In some embodiments, the escalating includes contacting a medical professional, e.g., electronically, to alert them of the subject's health condition.

In some embodiments, the respective preexisting health condition is selected from the group consisting of diabetes, prior bariatric surgery, and hypertension.

In some embodiments, the method further includes during an initial meeting, a primary follow-up meeting, or a secondary follow-up meeting, determining whether the subject has experienced a crisis event affecting the subject's physical or mental health. When it is determined that the subject has experienced the crisis event, escalating the subject's care to a professional according to a crisis management plan corresponding to the crisis event. In some embodiments, the escalating contacting, or instructing the subject to contact, a medical professional, adult protective service, mental health help lines, or police/911 to alert them of the subject's crisis.

In some embodiments, the method further includes, when the subject has been diagnosed with a respective preexisting health condition in a plurality of preexisting health conditions, receiving an approval from a medical professional that the subject may exercise. The method further includes determining, based on the respective preexisting health condition and whether the subject has experienced any symptoms of a plurality of symptoms during exercising, whether the subject has any limitations on ability to exercise, educating the subject on a type and amount of exercise that would support the management of the preexisting health condition, and facilitating subject creation of an exercise plan. The exercise plan includes at least one action step (e.g., define and review goals and actions steps 130, 140) for the subject to take in furtherance of achieving the long or short term goal.

In some embodiments, the method further includes, prior to obtaining from the subject at the first time point the first scored self-assessment, a referral (e.g., perform initial appointment, referral 110) from a health care provider associated with the subject, the referral identifying the health condition of the subject needing management.

In some embodiments, the method further includes providing (e.g., store notes 170, review notes and provide recommendations 172) electronic data relevant to the management of the health condition of the subject to a health care provider associated with the subject. The electronic data includes at least the long term goal for improving the health of the subject, the short term goal for improving the health of the subject, the at least one action step for the subject to take in furtherance of achieving the long or short term goal, and notes from a respective secondary follow-up meeting in the series of follow-up meetings.

In some embodiments, the method further includes receiving instructions (e.g., review notes and provide recommendations 172) from the health care provider associated with the subject to modify the long term goal for improving the health of the subject, the short term goal for improving the health of the subject, or the at least one action step for the subject to take in furtherance of achieving the long or short term goal.

In some embodiments, the method further includes obtaining, from the subject at a third time point occurring after the first time point and prior to the second point (e.g., at or near the end of the first epoch), a scored wellness satisfaction survey comprising a scored self-assessment of, for each respective health-related category in the plurality of health-related categories, a second measure of the subject's satisfaction with their own health relative to the respective health-related category. (e.g., complete program and wellness satisfaction surveys 175).

In some embodiments, the method further includes obtaining, from the subject at a fourth time point occurring after the first time point and prior to the second time point (e.g., between the third time point and the second time point), a program satisfaction assessment (e.g., complete program and wellness satisfaction surveys 175). The program satisfaction assessment includes a scored assessment of the subject's own health, an assessment whether the subject has gained any benefit from the method of managing their health condition between the first time point and the fourth time point, an assessment whether the subject would recommend the method of managing the health condition to another subject, and an assessment whether the subject would improve one or more features of the method of managing the health condition.

In some embodiments, the plurality of health-related categories includes a first category associated with physical health, a second category associated with nutritional health, a third category associated mental health, a fourth category associated with social health, a fifth category associated with the subject's environmental health, a sixth category associated with the subject's life purpose, and a seventh category associated with the subject's spiritual health.

In some embodiments, the first epoch is a month (e.g., 30-day goals in Table 1) and the second epoch is six months (e.g., 6-month goals in Table 1)

In some embodiments, facilitating subject creation of the health vision includes guiding the subject to identify the one or more values (e.g., develop health vision plan 172). The subject considers the one or more values to be most important in the subject's life. Facilitating subject creation of the health vision also includes guiding the subject to identify a connection between the one or more values and the health condition in the subject thereby ensuring that the created health vision plan for improving the health of the subject corresponds to the one or more values that the subject considers to be most important in the subject's life.

In some embodiments, facilitating subject creation of the health vision (e.g., develop health vision plan 172) includes guiding the subject to identify the one or more personal experiences. The subject associates the one or more personal experiences with feeling of success and engagement. Facilitating subject creation of the health vision also includes guiding the subject to identify one or more strengths that the subject utilized to achieve the one or more personal experiences that the subject associates with feeling of success and excitement.

In some embodiments, facilitating subject creation of the health vision (e.g., develop health vision plan 172) includes guiding the subject to identify the one or more behaviors. The one or more behaviors include behaviors that the subject is currently doing successfully to manage the health condition of the subject.

In some embodiments, facilitating subject creation of the health vision (e.g., develop health vision plan 172) includes guiding the subject to identify the one or more motivations by guiding the subject to identify and evaluate importance of the management the health condition of the subject.

In some embodiments, facilitating subject creation of the health vision (e.g., develop health vision plan 172) includes guiding the subject to create the path between the subject's current health and the subject's desired health with respect to the selected subset of health-related categories. The creating includes identifying a difference between the subject's health condition at the first time point and the subject's health condition in the vision, identifying a best case scenario and a worst case scenario for the subject's path, identifying perceived obstacles on the path, identifying the subject's support network, and identifying perceived strengths of the subject.

In accordance with some embodiments, a method for managing a health condition in a subject includes any of the features recited above.

The previous description is provided to enable any person skilled in the art to practice the various aspects described herein. While the foregoing has described what are considered to be the best mode and/or other examples, it is understood that various modifications to these aspects will be readily apparent to those skilled in the art, and the generic principles defined herein may be applied to other aspects. Thus, the claims are not intended to be limited to the aspects shown herein, but is to be accorded the full scope consistent with the language claims, wherein reference to an element in the singular is not intended to mean “one and only one” unless specifically so stated, but rather “one or more.” Unless specifically stated otherwise, the terms “a set” and “some” refer to one or more. Pronouns in the masculine (e.g., his) include the feminine and neuter gender (e.g., her and its) and vice versa. Headings and subheadings, if any, are used for convenience only and do not limit the embodiments disclosed herein.

It is understood that the specific order or hierarchy of steps in the processes disclosed is an illustration of exemplary approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

Terms such as “top,” “bottom,” “front,” “rear” and the like as used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

A phrase such as an “aspect” does not imply that such aspect is essential to the subject technology or that such aspect applies to all configurations of the subject technology. A disclosure relating to an aspect may apply to all configurations, or one or more configurations. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. A phrase such an embodiment may refer to one or more embodiments and vice versa.

The word “exemplary” is used herein to mean “serving as an example or illustration.” Any aspect or design described herein as “exemplary” is not necessarily to be construed as preferred or advantageous over other aspects or designs.

All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for.” Furthermore, to the extent that the term “include,” “have,” or the like is used in the description or the claims, such term is intended to be inclusive in a manner similar to the term “comprise” as “comprise” is interpreted when employed as a transitional word in a claim.

1. A method for managing a health condition in a subject in need thereof by reinforced lifestyle adjustment based on one or more underlying motivations, the method comprising:
obtaining, from the subject at a first time point, a first scored self-assessment for a plurality of health-related categories across a plurality of metrics, the first self-assessment including, for each respective health-related category in the plurality of health-related categories:
a first measure of the subject's assessment of their own health relative to the respective health-related category,
a first measure of the subject's satisfaction with their own health relative to the respective health-related category, and
a first measure of the subject's desire to improve their own health relative to the respective health-related category;
selecting a first subset of health-related categories from the plurality of health-related categories based on the first scored self-assessment;
facilitating subject creation of a health vision plan for improving the health of the subject with respect to the selected subset of health-related categories using first information arising from one or more meetings with the subject, wherein the first information comprises:
a description of the subject's desired health outcome elicited from the subject, and
one or more values, one or more personal experiences, one or more behaviors, or one or more motivations of the subject elicited from discussion with the subject,
establishing a short term goal for improving the health of the subject, relative to the selected subset of health-related categories, over a first epoch,
establishing a long term goal for improving the health of the subject, relative to the selected subset of health-related categories, over a second epoch, wherein the second epoch subsumes the first epoch and is at least twice as long as the first epoch, and the short term goal and the long term goal create a path between the subject's current health and the subject's desired health with respect to the selected subset of health-related categories,
identifying one or more strategies the subject believes will be effective to achieve the short or long term goals;
identifying, based on at least the one or more identified strategies, at least one action step for the subject to take in furtherance of achieving the short or long term goal;
re-evaluating the at least one action step using second information obtained from a primary follow-up meeting with the subject, wherein the follow-up meeting occurs after one or more initial meetings and during the first and second epochs, and wherein the second information comprises:
a description of the subject's perception of their execution of the at least one action step elicited from the subject,
a determination of whether the subject wishes to modify the long term goal, the short term goal, or the at least one action step, wherein
when it is determined that the subject wishes to modify the long term goal, the short term goal, or the at least one action step, the long term goal, the short term goal, or the at least one action step is reestablished in modified form;
re-evaluating the at least one action step during each of a series of secondary follow-up meetings with the subject, wherein the series of secondary follow-up meetings occur after the primary follow-up meeting and during the second epoch; and
providing to the subject, outside of a meeting with the subject, one or more messages that include content configured to (i) encourage the subject to achieve the long term goal or the short term goal, (ii) remind the subject of one or more of the at least one action step, and/or (iii) educate the subject on the selected subset of health-related categories.

2. The method of claim 1, further comprising:
obtaining, from the subject at a second time point occurring after the first time point, a second scored self-assessment for the plurality of health-related categories across the plurality of metrics, the second scored self-assessment including, for each respective health-related category in the plurality of health-related categories:
a first measure of the subject's assessment of their own health relative to the respective health-related category,
a second measure of the subject's satisfaction with their own health relative to the respective health-related category, and
a second measure of the subject's desire to improve their own health relative to the respective health-related category; and
selecting a second subset of health-related categories from the plurality of health-related categories based on the second scored self-assessment.

3. The method of claim 1, further comprising:
monitoring one or more health parameters of the subject, the monitoring including:
receiving, at a first frequency, respective records of the one or more health parameters of the subject at an electronic device, and
reviewing the respective records received for the one or more health parameters.

4. The method of claim 3, wherein the monitoring the one or more health parameters includes monitoring the subject's blood pressure, weight, step count, movement, heart rate, blood glucose level, blood cholesterol, food intake, drink intake, and/or notes regarding the subject's emotional state.

5. The method of claim 3, wherein the electronic device is a health monitoring device and the respective records are automatically generated upon a measurement performed by the health monitoring device.

6. The method of claim 3, wherein the respective records are input manually by the subject on a client electronic device.

7. (canceled)

8. The method according to claim 3, further comprising:
prior to a respective secondary follow-up meeting in the series of secondary follow-up meetings, determining whether the monitored one or more health parameters indicate that the subject is taking the at least one action step in furtherance of achieving the short or long term goal; and
while re-evaluating the at least one action step during the respective secondary follow-up meeting:
when the monitored one or more health parameters indicate that the subject is taking the at least one action step in furtherance of achieving the short or long term goal, discuss with the subject (i) why the subject has been successful in taking the at least one action step, and (ii) whether to adjust the at least one action step to further support achievement of the short or long term goal; and
when the monitored one or more health parameters indicate that the subject is not taking the at least one action step in furtherance of achieving the short or long term goal, discuss with the subject (i) why the subject may not be taking the at least one action step, and (ii) whether to adjust the at least one action step to further encourage the subject to take the at least one action step.

9. The method according to claim 3, further comprising:
prior to a respective secondary follow-up meeting in the series of secondary follow-up meetings, determining whether the monitored one or more health parameters indicate that the subject is progressing towards achievement of the short term goal or the long term goal; and
while re-evaluating the at least one action step during the respective secondary follow-up meeting:
when the monitored one or more health parameters indicate that the subject is progressing towards achievement of the short term goal or the long term goal, discuss with the subject (i) why the subject has been successful in progressing towards achievement of the short term goal or the long term goal, and (ii) whether to adjust the short term goal or long term goal to further improve the health of the subject; and
when the monitored one or more health parameters indicate that the subject is not progressing towards achievement of the short term goal or the long term goal, discuss with the subject (i) why the subject may not be progressing towards achievement of the short term goal or the long term goal, and (ii) whether to adjust the at least one action step in furtherance of achieving the short term goal or the long term goal.

10. The method according claim 1, wherein:
the description of the subject's perception of their execution of the at least one action step elicited from the subject comprises:
a description of a positive event that has occurred with respect to the subject's health condition since a previous meeting, and
an identification of a strength that facilitated the positive event; and
the determination of whether the subject wishes to modify the long term goal, the short term goal, or the at least one action step is based on at least the subject's perception of their execution of the at least one action step.

11. The method according to claim 3, wherein the content of the one or more messages provided to the subject outside of a meeting with the subject is based on at least the review of the records received for the one or more health parameters.

12. The method according to claim 1, wherein at least one message of the one or more messages provided to the subject outside of a meeting with the subject is automated and is provided at a predetermined time.

13. The method according to claim 1, further comprising, when the subject has been diagnosed with a respective preexisting health condition in a plurality of preexisting health conditions:
determining, based on the reviewing the respective records received for the one or more health parameters, whether the subject has experienced a triggering event associated with the respective preexisting health condition; and
when it is determined that the subject has experienced the triggering event, escalating the subject's health condition to a medical professional according to an escalation plan corresponding to the preexisting health condition.

14. The method of claim 13, wherein the escalating includes contacting the subject and providing the subject recommendation for addressing the triggering event associated with the respective preexisting health condition.

15. The method according to claim 1, further comprising, when the subject has been diagnosed with a respective preexisting health condition in a plurality of preexisting health conditions:
during a respective secondary follow-up meeting in the series of follow-up meetings, determining whether the subject has experienced one or more symptom of a triggering event associated with the respective preexisting health condition; and
when it is determined that the subject has experienced the triggering event, escalating the subject's health condition to a medical professional according to an escalation plan corresponding to the preexisting health condition.

16. The method according to claim 13, wherein the respective preexisting health condition is selected from the group consisting of diabetes, prior bariatric surgery, and hypertension.

17. The method according to claim 1, further comprising:
during an initial meeting, a primary follow-up meeting, or a secondary follow-up meeting, determining whether the subject has experienced a crisis event affecting the subject's physical or mental health; and
when it is determined that the subject has experienced the crisis event, escalating the subject's care to a professional according to a crisis management plan corresponding to the crisis event.

18. The method according to claim 1, further comprising, when the subject has been diagnosed with a respective preexisting health condition in a plurality of preexisting health conditions:
receiving an approval from a medical professional that the subject may exercise;
determining, based on the respective preexisting health condition and whether the subject has experienced any symptoms of a plurality of symptoms during exercising, whether the subject has any limitations on ability to exercise;
educating the subject on a type and amount of exercise that would support the management of the preexisting health condition; and
facilitating subject creation of an exercise plan, wherein the exercise plan includes at least one action step for the subject to take in furtherance of achieving the long or short term goal.

19. (canceled)

20. The method according to claim 1, further comprising providing electronic data relevant to the management of the health condition of the subject to a health care provider associated with the subject, wherein the electronic data includes at least the long term goal for improving the health of the subject, the short term goal for improving the health of the subject, the at least one action step for the subject to take in furtherance of achieving the long or short term goal, and notes from a respective secondary follow-up meeting in the series of follow-up meetings.

21. The method according to claim 1, further including:
obtaining, from the subject at a fourth time point occurring after the first time point and prior to the second time point, a program satisfaction assessment from the subject, the program satisfaction assessment including:
a scored assessment of the subject's own health,
an assessment whether the subject has gained any benefit from the method of managing their health condition between the first time point and the fourth time point;
an assessment whether the subject would recommend the method of managing the health condition to another subject, and
an assessment whether the subject would improve one or more features of the method of managing the health condition.

22-23. (canceled)

24. The method according to claim 1, wherein establishing the short term goal and the long term goal includes:
guiding the subject to create the path between the subject's current health and the subject's desired health with respect to the selected subset of health-related categories by:
identifying a difference between the subject's health condition at the first time point and the subject's health condition in the vision,
identifying a best case scenario and a worst case scenario for the subjects path,
identifying perceived obstacles on the path,
identifying the subject's support network, and
identifying perceived strengths of the subject.
